(19) United States
(12) Patent Application Publication
(10) Pub. No.: US 2021/0137441 A1
(43) Pub. Date: May 13, 2021

(54) EQUIPMENT FOR DETERMINING HUMAN HEALTH STATUS USING SYNCHRONIZED DATA FROM MULTIPLE SENSORS AND METHOD OF OPERATION THEREOF

(71) Applicant: UAB METAPRO HOLDING, Vilnius (LT)

(72) Inventor: Albinas STANKUS, Kaunas (LT)

(21) Appl. No.: 16/649,033

(22) PCT Filed: Sep. 20, 2018
(86) PCT No.: PCT/IB2018/057242

§ 371 Date: Mar. 19, 2020

(30) Foreign Application Priority Data
Sep. 22, 2017 (LT) 2017527

(51) Int. Cl. A61B 5/352; G16H 40/67; G06F 16/27; G16H 50/20; A61B 5/339; A61B 5/308

(52) U.S. Cl. CPC A61B 5/352; G16H 40/67; G06F 16/27; A61B 2560/0242; A61B 5/339; A61B 5/308; G16H 50/20

(57) ABSTRACT

A new measurement, analysis and diagnostic equipment is provided for use in a medical field for measuring and analyzing cardiac activity. Measured parameters of human and environmental factors are synchronized in accordance with cardiac activity parameters (Recg or RRI). The measurement, analysis and diagnostic equipment includes at least a central processing unit and sensors. Sensors process the measured data and transmit only a significant feature or sequence over time of features to the central unit, namely, they provide already processed data. In this way, technical resources of the central unit can be used to process and deliver the data in real time, to perform an advanced analysis, to include a large amount of the measured parameters.

### FIELD OF THE INVENTION

The invention relates to the field of medical equipment, and in particular, equipment for measuring, processing parameters of factors affecting human health and method of operation thereof.

### DESCRIPTION OF THE RELATED ART

This invention provides new equipment used in a medical field for measuring, analyzing parameters of the human being and his environmental factors and diagnosing health status, which synchronizes the measured parameters in accordance with the parameters of cardiac activity. Sensors of the equipment have a two-way communication with a central processing unit and are intended to process the measured data by providing only a significant feature(s) or sequence of the feature (s) to the central unit, without having to use resources of the central unit to process the insignificant data.

Document U.S. Pat. No. 6,908,437 (B2), published on 21 Jun. 2005, provides a system and method for diagnosing and monitoring heart failure. The system has a database where all the data collected from sensors is stored, the server processes these data. In this system, the server is heavily loaded, since it has to process a very large amount of data. From functions performed by the server, we see that a real-time reporting of analysis data and diagnosing is not possible. Also, the document does not mention anything about the synchronization of measurement data and the main feature extraction.

Document U.S. Pat. No. 5,590,648 (A), published on 7 Jan. 1997, describes a personal healthcare system whose data processing unit has a two-way electrical connection with sensors. It can not only receive data from sensors, but also send them control commands. In the case of a system provided in the document, it does not mention anything about the ability of sensors to process the measurement data, as well as to extract the main feature.

Summarizing the documents of the related art, the following deficiencies can be distinguished:

The entire data processing process occurs in the central processing unit, which causes it to be very loaded;
There is no possibility to measure and analyze the data in real time;
The measurement data is not synchronized in accordance with the synchronizing signal;
The main feature (s) or their sequence over time is not extracted from the measurement data;
Due to the different nature of the measured processes, it is difficult to perform the analysis on links among the measured measurement data.

This invention provides a technical solution that does not have the above deficiencies.

### SUMMARY OF THE INVENTION

In order to diagnose human health as precisely as possible, it is necessary to measure and analyze as much as possible human and his environmental factors. Due to an increasing amount of measured environmental factors and increasing amount of data, it is difficult for medical professionals to choose the factors necessary for measurement, to properly configure the measurement, analysis, and diagnostic equipment.

The measurement, analysis and diagnostic equipment is composed of at least a central processing unit and sensors. Unlike the related art, sensors process the measured data and transmit only a significant feature or sequence over time of features to a central unit, i.e. they provide already processed data. In this way, technical resources of the central unit can be used to process and deliver the data in real time, to perform an advanced analysis, to include a large amount of the measured parameters. One of the main tasks of the central unit is to measure existing relations (links) between sequences of significant features using multivariate frequentative analysis methods that require precise synchronization among processes being recorded. Health diagnosing is not possible without this task.

This invention is a new measurement, analysis and diagnostic equipment used in a medical field for measuring and analyzing parameters of cardiac activity and subsystems related to functions thereof. Parameters of human and his environmental factors measured are synchronized in accordance with cardiac activity parameters (Recg or RRI). The measurement, analysis and diagnostic equipment is composed of at least a central data processing unit and sensors. Unlike the related art, sensors process the measured data and transmit only a significant feature or sequence over time of features to a central unit, i.e. they provide already processed data. In this way, technical resources of the central unit can be used for data processing and real-time delivery, for the purpose of performing a higher level of analysis, to include a large amount of measured parameters. One of the main tasks of the central unit is to measure existing relations (links) between sequences of significant features using multivariate frequentative analysis methods that require precise synchronization among processes being recorded. Health diagnosing is not possible without this task.

### BRIEF DESCRIPTION OF THE DRAWINGS

FIG. 1 provides a principal scheme of the equipment with information communications.

FIG. 2 provides a principal scheme of the equipment with a synchronizing sensor.

FIG. 3 provides a scheme of signal and data processing.

The presented figures are more illustrative, scale, proportions and other aspects do not necessarily correspond to a real technical solution.

### DETAILED DESCRIPTION

The parameters of human cardiac activity are related not only to the rhythm of human physical activity during the day, but also to the multidimensional effects of central and peripheral clocks through the nervous system to cardiac activity. Currently, circadian rhythm disorders are found to be associated with an increased risk of cardiovascular morbidity and mortality. In order to accurately diagnose the disease, to determine the actual health status, the daily balance between the active and passive state of the body is often unknown. Tiredness, anxiety or depression that may occur may be related to circadian rhythm disorders. Identification of these phenomena is particularly relevant for diagnosis and prophylaxis of diseases, achieving sport outcomes, assessing rest and effect of stress, sleep quality, choosing work, assessing the degree of energy consumption and recovery, and avoiding sudden disorders in the body. The example given clearly illustrates the fact that the accurate determination of the human health status is affected by a number of factors (both human internal and environmental) and their links. The more factors are measured, the more links of the measured factors are determined, the more accurate the health status can be diagnosed and the methods of treatment can be chosen more efficiently. The objective is to objectively measure and use the following processes affecting the human body and their components for diagnosing:

a) physical activity (body strength, body composition, flexibility, speed, endurance, etc.);
b) mental and psychological processes (attention research, language modulation, teaching process, loud reading, computerized text typing, effects of art (senses and actions of the person in rehabilitation and elsewhere), behavioral graph (Action-habit-character formation), mental fatigue);
c) spiritual capacities (seeking of knowledge, need for beauty, seeking justice, love problems (affection), emotional fatigue, fear);
d) social capacity (reading of announcements in the hall, playing of music and singing, sporting events, family behavior, restrictions on people with special needs (motor, sensory, mental), ergonomic, reproductive function).

On the other hand, a large amount of measured factors leads to large amounts of data, which results in the problem of data processing, selection of the feature and information of proper processes. The large amounts of data processed result in long-lasting data processing technical processes. In the case of the medical equipment, ultra-fast data processing and delivery in real time is critically important. Due to a large number of various process measurement results, it is difficult for a medical professional to select the necessary measurements, difficult to group the measured data, and configure the measurement equipment in such a way as to make significant findings from the data provided, relevant to diagnosing.

This invention provides the equipment for assessing the human health status, and especially for cardiac activity, and the measured parameters are synchronized in accordance with cardiac activity. Considering that all the measured parameters are synchronized in accordance to cardiac activity, such equipment is more suitable for determination of cardiac activity parameters, however, if the synchronized signal is chosen other than the cardiac activity parameter, the equipment provided in the present invention can be adapted for other measurements of the human health status. The equipment is composed of a number of sensors that can not only measure the parameters, but also process the measured data in accordance with the rules defined by the central unit.

The measurement, analysis and diagnostic equipment provided in the present invention is composed of at least the following essential parts:

a central information processing unit (central unit) (1) (FIG. 1) (FIG. 2) for controlling sensors (2), exchanging the data with sensors (2), processing information, providing information in the format suitable for displaying to the human being;

sensors (2), (2.1) for measuring human and his environmental parameters, for processing the data of parameters in accordance with the rules defined by the central unit (1), for transmitting the processed data to the central unit (1).

In addition to the above-mentioned parts composing the equipment, there are additionally other parts as usual for such equipment: the communication means among internal components of the equipment and with external components of the equipment, memory modules and other parts.

In the present invention, the term “sensor”, in contrast to the related art, refers to a unit that not only converts the measured physical parameter value to the corresponding value of the electrical signal, but also processes the signal of the measurement result. In the present invention, the processing can be divided into two conditional parts, wherein in the first part the primary electrical signal is amplified, if necessary, it is converted to a different electrical signal (e.g. from analogue to digital), i.e. in the first part the primary electrical signal is converted into data suitable for further processing. The second part of processing of the electrical signal of the measured parameter is the filtering of the signal, noise elimination, data processing, significant feature extraction. Data processing is used to find the significant feature of the measured parameter and/or sequence of features over time. In the present invention, the significant feature is considered as the result of the processed information of the measured parameters for aiming that the mentioned result transmitted to the central unit (1) is sufficiently processed in the sensor (2) in order not to load the operation of the central unit (1), but the data value must be retained for processing throughout the research. In each particular measurement case, the significant feature of the measurement may be different in relation to the entire measurement objective, the results to be obtained.

Each sensor (2) in the present invention is autonomous, having a transformer of the measured parameter value to the electrical signal, electrical signal amplifier, analog to digital signal converter, data processing unit, operative and permanent memory modules, communication modules for receiving information from other devices, and sending and to other elements for independent, autonomous operation. The software for the data processing, the significant feature extraction can be pre-installed into the sensor (2).

In the present invention, sensors (2) have a two-way communication for transmitting information, i.e. the sensor (2) can not only transmit the results of the measurement to the central unit (1), but also receive control rules from the sensor (2) of the central unit (1), which may include the specific significant feature that the sensor (2) should transmit to the central unit (1). In this way, a significant part of data processing is performed in the sensor (2) measuring every parameter, and only the data relevant to the specific measurement reaches the central unit (1), which the central unit (1), if necessary, can further process. Transferring a large part of technical capacity for data processing from the central unit (1) to sensors (2) large capacities in the central unit (1) are released. In this way, the central unit (1) can allocate its capabilities to significant, more sophisticated calculations to achieve advanced results, for example, to determine the trends of health status, to select the treatment method and to monitor the outcome of the method implementation, to compare results with other similar results and/or others.

All of the human internal processes and external environmental processes affecting them, taking into account cardiac activity periods, can be grouped into three main groups:

(a) processes that have the action period very close to the cardiac action period (e. g. ECG, systole, hemodynamic parameters, etc.);
(b) processes that have the action period significantly shorter than the cardiac activity period (muscle and brain activity, etc.);
(c) processes that have the action period significantly longer than the cardiac activity period (breathing, blood oxygenation, etc.).

According to the nature of their repetition, these external and internal processes can be attributed to determined processes, the analysis of which involves a structural analysis.

The mentioned measured parameters of internal and external factors affecting the human being according to their temporal characteristics are processed by the following methods: a) averaging, b) integration, c) structural analysis. In other embodiments, the combination of the above processing methods may be applied, and due to metrological requirements, additional mathematical operations may be applied. The processing of the measurement data by these methods is performed in sensors (2). Multivariate frequentative methods are used to process the synchronized data in the central unit (1), which results in a more accurate level of health diagnosing.

In the present invention, one of the functions of the sensors (2) is to synchronize the measurement data of different parameters on a time scale, to equalize the time scales. One of the functions of the synchronization on a time scale when examining the cardiovascular system is the selection of ECG R denticle (Recg) as a synchronizing signal. When the electrocardiogram detects an R denticle, a synchronizing signal is sent from the central unit (1) to the sensors (2). The sensors (2), in accordance with the synchronizing signal, configure the temporal characteristics of the measured parameters. In one embodiment of the invention, the measured parameters with synchronized time characteristics can be transmitted to the central unit (1), which synchronizes moments and periods of the measurement data of different parameters on the time scale, by equalizing time scales. In other embodiments, the measured parameter with the synchronized time characteristic in accordance with the rules defined by central unit (1) can be further processed to reduce the amount of data transmitted to the central unit (1) and to concentrate more relevant data into the information transmitted to the central unit (1).

The objective of the entire measurement equipment is to find and access the links of the measured parameters.

In the present invention, all the measured signals are divided into the synchronizing (master) and synchronized (slave).

In another embodiment of the invention, a special synchronizing signal (Recg or RRI) sensor (2.1), differed from the other sensors (2), is used to transfer even greater computing capacity from the central unit (1). In this case, the mentioned synchronizing signal sensor (2.1) sends a synchronization signal to other sensors (2), in accordance with which other sensors (2) synchronize the measurement data, and also receive a significant feature (s) or sequence (s) over time thereof from the other sensors (2). In this way, significant features and/or their sequences over time from the synchronizing signal sensor (2.1) processed by all sensors (2), (2.1) get in the central unit (1).

In one of the embodiments, the data processing schema and sequence may be as shown in FIG. 3. Depending on the temporal characteristics of the measured parameter, as mentioned, the processes are divided into three different groups, synchronized in accordance with the Recg signal. The synchronized data is subjected to appropriate signal and data processing methods: averaging, integration, structural analysis and additional operations. The sensors (2) divide the processes in accordance with their temporal characteristics, synchronize, process (extract the significant feature (s) (or sequence (s) over time) and transmit it to the central unit (1).

This invention provides the diagnostic equipment which instead of the direct record and demonstration of parameters, providing the processed measurement data, proceeds to the extraction of physiologically significant features and capture of their dynamics in the time scale. As mentioned, one of the basic conditions for the proper significant feature extraction is the formation of a synchronizing signal, the transmission to the sensors (2), which synchronize all the measured processes in accordance with the synchronizing signal. As mentioned, the synchronizing signal may be the appearance of the peak time point of the electrocardiogram R (Recg). Another possible synchronizing signal is the duration of the RR interval (usually the duration is 0.25-2 s.) or even a repetitive time period that includes at least one Recg.

In other embodiments, in accordance with the rules defined by the central unit (1), the sensors (2) can generate not only one, but some significant features or time sequences of significant features after the data processing.

In other embodiments, the central unit (1) can perform a function of the link between the entire measurement unit and the human function, i.e. to display results of the measured parameters, to provide the measured results in accordance with the rules defined by the human being, to synchronize the displayed results in accordance with the parameters of cardiac activity. Another part of the interface of the unit with the human being is the information entry device. One of the appropriate ways to enter information is visual information submission devices with touch-controlled parameters for changing demonstration and control modes.

In many cases, medical professionals do not have deep technical competencies for complex configurations of the measurement equipment and changes to settings, thus in this case, one of the most important characteristics of the measurement equipment is the ability to easily identify and configure the measurement equipment with a simple user interface. In other embodiments, setting and configuration of parameters can be performed at two levels, when more complex changes to parameters and configurations of the user interface are performed by specialists having technical competence, while simpler changes using a user interface are left to medical professionals.

The measured parameters and sensors (2) measuring them are selected in accordance with already existing links of parameters established and validated by science, the result to be measured. This is the mode of operation of the measurement equipment wherein the measurement equipment is used for diagnostic purposes only. In this case, the predefined rules for using the measurement equipment are used; the user interface can be used with predefined settings and/or set of settings. In the other case of using the measurement equipment, it is possible to try to measure new parameters, to extract new significant features, and to determine new significant links of parameters, i. e. the equipment is used for new cognitive, scientific researches. In both of the cases described above, different user interfaces may be used: a simpler, with predefined settings for normal diagnosis and an interface for research, where the researcher has the ability to select a larger amount of equipment settings.

One of the main functions of the data processing of the equipment provided in this invention is to assess the degrees of links and times of interaction of the examined processes, and proper data processing compatibility, calibration throughout the data channel from the sensor (2) to the central unit (1) must be ensured. Particular attention is paid to the amplitude and phase characteristics of the sensors (2) and the central unit (1). Their sensitivity and modality must accurately reproduce the recorded physiological process. Sensors (2) should not block the nature of the process itself (for example, the photoplethysmogram sensor often compresses capillaries). It is advisable to avoid preliminary processing of data on physiological processes or their transformations, if it can provide information with additional artificial addictions (e. g. hyperbolic, changing the RR interval to the pulse rate per minute).

In order to illustrate and describe the invention, the description of the preferred embodiments is presented above. This is not a detailed or restrictive description to determine the exact form or embodiment. The above description should be viewed more than the illustration, not as a restriction. It is obvious that specialists in this field can have many modifications and variations. The embodiment is chosen and described in order to best understand the principles of the present invention and their best practical application for the various embodiments with different modifications suitable for a specific use or implementation adaptation. It is intended that the scope of the invention is defined by the definition added to it and its equivalents, in which all of these definitions have meaning within the broadest limits, unless otherwise stated.

In the embodiments described by those skilled in the art, modifications may be made without deviating from the scope of this invention as defined in the following definition.

1. An equipment for determining human health, having:
a central information processing unit for controlling sensors, exchanging data with sensors, processing information, providing information in a format suitable for displaying to a human being;
sensors for measuring human and environmental parameters,
wherein the sensors divide the processes in accordance with their temporal characteristics, synchronize, process data in accordance with rules defined by the central unit, after processing the data, a significant feature and/or sequence of features over time of the parameter is transmitted to the central unit, where the significant feature is considered as the result of the processed information of the measured parameters for aiming that the mentioned result transmitted to the central unit is sufficiently processed in the sensor in order not to load the operation of the central unit.

2. The equipment for determining human health according to claim 1, wherein each sensor in the present invention is autonomous, having its transformer of the measured parameter value to the electrical signal, electrical signal amplifier, analog to digital signal converter, data processing unit, operative and permanent memory modules, communication modules for receiving information from other devices and sending and other elements for independent, autonomous operation.

3. The equipment for determining human health according to claim 1, wherein, in accordance with the rules defined by the central unit, sensors can generate not only one, but some significant features or sequences over time of significant features after the data processing.

4. The equipment for determining human health according to claim 1, wherein the equipment has a synchronizing signal sensor that sends a synchronization signal to other sensors, according to which other sensors synchronize the measurement data, and also receives the significant feature(s) or its sequence over time from other sensors, transmits the collected data to the central unit.

5. The equipment for determining human health according to claim 1, wherein the central unit (1) synchronizes moments and periods on time scale of the measurement data of different parameters by equalizing time scales.

6. A method for processing the parameters affecting human health using the equipment according to claim 1, wherein the measured signals of factors affecting human health are synchronized in accordance with the Recg signal.

7. The method for processing the parameters affecting human health according to claim 6, wherein a synchronizing signal is a RR interval or repetitive time period, which includes at least one Recg.

8. The method for processing the parameters affecting human health according to claim 6, wherein an averaging, integration, structural analysis, or combination of these methods with meteorological conversion is used for the processing of the synchronized data in sensors.
